(19) United States
(12) Patent Application Publication
(10) Pub. No.: US 2010/0068279 A1
(43) Pub. Date: Mar. 18, 2010

(54) BURN BANDAGE

(75) Inventor: Ryan Hartwell, Shanty Bay (CA)

Correspondence Address: OCCHIUTI ROHLICEK & TSAO, LLP, 10 FAWCETT STREET, CAMBRIDGE, MA, 02138, US

(73) Assignee: ATS Biotech Inc., Oakville (CA)

(21) Appl. No.: 12/516,674

(22) PCT Filed: Nov. 28, 2007
(86) PCT No.: PCT/CA2007/002140

§ 371 Date: Oct. 14, 2009

Related U.S. Application Data
(60) Provisional application No. 60/861,250, filed on Nov. 28, 2006.

(51) Int. Cl. A61K 9/14; A61P 17/02

(52) U.S. Cl. 424/486

(57) ABSTRACT

The present invention provides a novel dressing for the treatment of skin injuries, particularly burns. The device comprises an gel-like interpenetrating network comprising polyvinyl alcohol and polyaspartate. Additional natural and synthetic components may be added to the network or to the surface of the device. The device provides cooling, protection from infection and delivery of therapeutic agents.

### FIELD OF INVENTION

The present invention relates to the treatment of burns. In particular, the invention relates to a hydrogel dressing that may be adapted for delivery of therapeutics to burn sites.

### BACKGROUND OF THE INVENTION

The skin is the body's largest organ and performs many vital functions, including protecting the body from invasion by bacteria and viruses. Burned skin not only loses its ability to protect against infection, it provides a favourable environment for the growth of microorganisms. A burn is any injury to the skin resulting from excess heat or cold chemicals, radiation or electricity. It is therefore important to find methods and compositions that promote healing of burned skin.

In a treatment of burns, bandages are used to protect against infection, reduce heat and water vapor loss from burned skin, protect the sensitive area, and help keep limbs, fingers and toes in a proper position for healing. In addition, certain bandages collect drainage from the wounds to promote healing.

Immediately after a burn, the blood vessels in the adjacent area are altered. There is initially an intense vasoconstriction followed by vasodilation during which the capillaries become more permeable. This results in abnormally high hydrostatic pressure gradients, which force intravascular fluid into the interstitial spaces. Free radical species, lipid peroxides, reactive oxygen and nitric oxide species attenuate the normal inflammatory responses and drive up blood pressure within the microvasculature. There is increased vascular permeability and interstitial pressure and the normal membrane polarity is disrupted. This leads to ischemia and lethal imbalances in the tissue.

All burns with the exception of mild first-degree burns should get immediate medical attention to reduce the risk of infection, dehydration and other possible complications. There remains a need for novel burn treatments that will reduce tissue damage and promote healing.

### SUMMARY OF THE INVENTION

The present invention provides a novel device composition and method for the improved treatment of skin injuries, particularly burns.

According to an aspect of the present invention there is provided a topical device for the treatment of a skin injury. The device comprises an interpenetrating network of polyvinyl alcohol (PVA) and polyaspertate (PASP). The PVA and PASP are partially cross-linked to form a hydrogen-bonded network.

In one preferred embodiment, the device comprises about 5-10% w/v PVA. In a more preferred embodiment, the device comprises 6-9% w/v PVA. In one preferred embodiment, the device comprises about 6% PVA and in another preferred embodiment it comprises about 9% PVA.

The device also comprises about 2-10% w/v PASP. In a preferred embodiment, the device comprises about 3-5% w/v PASP.

In yet another embodiment, the device also includes 6-10% glycerol or derivatives thereof, such as propylene glycol, polypropylene glycol, ethylene glycol, tryethylene glycol, polyethylene glycol, or other related substances.

In another preferred embodiment, the composition of the device includes antimicrobial agents to help prevent infection and contaminated.

In a further preferred embodiment, the device comprises an additive selected from the group consisting of L-ascorbic acid, alpha tocopherol, A. vera WLE, glutathione, cineol, and/or additional glycerol. It may also include L-glutamine, N-acetyl cysteine, epi-gallacto catechin gallate, IP6 and inositol derivatives, and lavender oil. These additives are preferably included in an amount of about 5% w/v.

In another aspect of the invention, a method of preparing a bandage for the treatment of a burn is provided. The method comprises preparing a base gel solution of PVA and PASP and combining the base solution with an additive such as A. vera WLE, L-ascorbic acid, alpha tocopherol, glutathione, and/or cineol.

In a preferred embodiment, the bandage is prepared by combining about 6-10% w/v PVA, 2-10% w/v PASP, and about 6-10% glycerol or a derivate thereof. These components form a base gel solution. In a further preferred embodiment, a 10% w/v PVA and 3% glycerol solution is prepared and then PASP is added to the solubilized solution. Once the gel is formed, it is cooled under vacuum for at least one hour to remove any trapped air bubbles. Additives such as L-ascorbic acid, alpha tocopherol, A. vera WLE, glutathione, cineol and glycerol can be added to the PVA/PASP gel-interpenetrating network. In another embodiment, these agents can be added to the gel surface after it has gelled, and with successive freeze/thaw cycles, can be absorbed and held within the gel bandage. In this method a final thin film of PVA can be poured onto the gel and allowed to dry securing the agents within.

The present invention has many advantages over the prior art. It enhances the removal of free radical reactive oxygen species, nitric oxide reactive species and free radicals. It also promotes cellular adhesion and differentiation and enhances immunological responses. The nature of the gel composition helps remove exudates and reduces ischemia and excess edema. The interpenetrating network comprising the PVA/PASP gel also can be used to carry other agents to replenish natural scavengers and proliferation agents. Furthermore, the bandage comprises a biocompatible hylauronic acid analogue (PVA) and a homo polymer, both of which have been shown to well suited for biomaterials. A further advantage of the invention is that it provides a barrier to prevent possible infection. Through the cooling effect, the device also helps to reduce temperature and pain.

### BRIEF DESCRIPTION OF THE DRAWINGS

FIG. 1 shows a hydrogel bandage in accordance with one embodiment of the invention;

FIG. 2 shows a hydrogel bandage in accordance with one aspect of the invention in use on a hand; and

FIG. 3 shows a micrcoscopic view of a bandage in accordance with one aspect of the invention.

### DETAILED DESCRIPTION

The novel burn device of the invention is composed of a self-sustaining interpenetrating network (IPN) of polyvinyl alcohol (PVA) and polyaspartate (PASP). These two water-soluble polymers, when partially cross-linked, form a hydrogen-bonded network in the gel state. The IPN is plasticized with glycerol and ionically cross-linked with tetraborohydrate, producing a gel at about pH 7.5. The plasticization with the glycerol (or one of its derivatives) allows for the gels to initially form as cryogels using freeze thaw cycles. A gel network of PVA, 5 PASP, and glycerol produces a translucent, thin elastomer bandage that can swell up to at least 500% of its dry weight. The network is also porous enough for drug delivery. The device may be embedded with both natural, therapeutic agents and synthetic agents. In one preferred embodiment, antioxidants which can diffuse from the bandage into the wound site to trap and eliminate reactive oxygen species and aid in re-epithelialization are included. It is an advantageous effect of the device that it acts as an analogue to hylauronic acid (PHA) and thereby provides an excellent substrate for cellular adhesion. In addition to this, homopolymers, such as polyaspartic acid have shown to be well tolerated and do not induce undesirable immune responses. A further advantage of the invention is that, as a hydrogel, the bandage is constantly releasing water vapor to the surroundings. The resultant latent heat exchange provided by this process cools the bandage to temperatures close to 22° C. This cooling effect provides patient comfort and promotes healing.

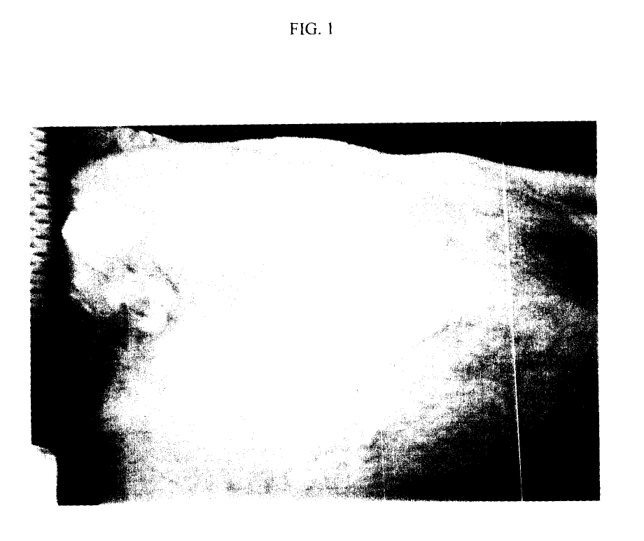

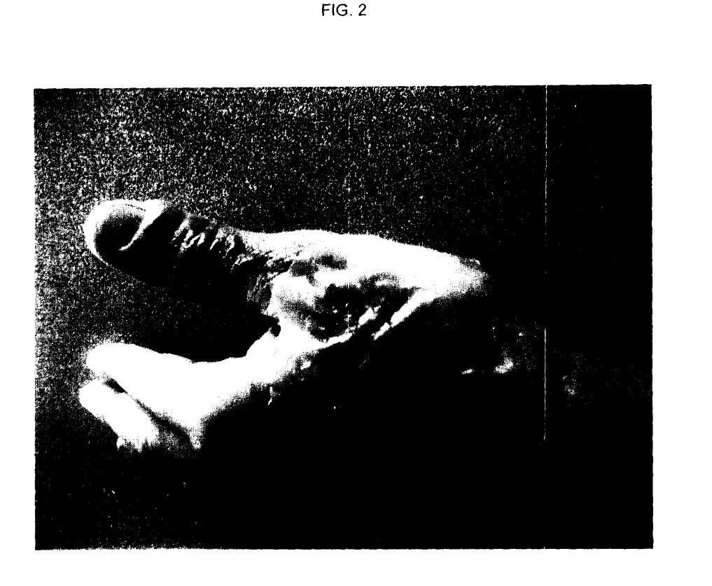

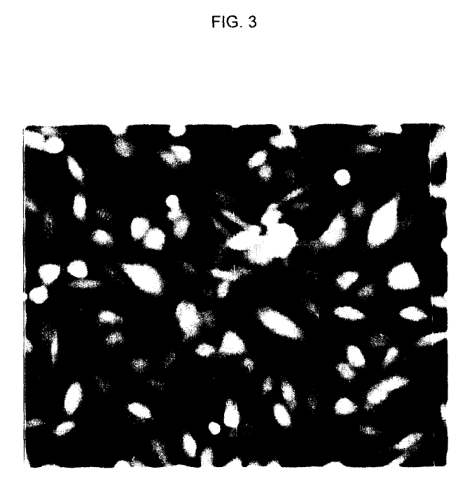

FIGS. 1 to 3 illustrate a bandage in accordance with particular embodiments of the invention. FIG. 1 shows a hydrogel bandage, FIG. 2 shows a hydrogel bandage applied to a hand and FIG. 3 shows the structure of a hydrogel in accordance with one aspect of the invention.

In one aspect of the invention, a device comprising an IPN of PVA and PASP is provided. These two components (PVA and PASP) provide for a stable hydrogel at room temperature without the use of a foreign substrate such as a foam or polyester. In a preferred embodiment, the polymer architecture is preferably then further gelled by the addition of A. vera whole leaf extract (A. vera WLE). As a gel itself, the A. vera produces a thickening effect within the uncured gel formulation. In addition to A. vera WLE natural products, essential oils and anti-oxidants may be added to the bandage. Other possible additives include biologics such as IL-1, IGF, KGF and EGF and/or fibroblasts from patients requiring skin grafting that may be added to the bandage for delivery and tissue re-growth within the wound site. Synthetic analogues of endogenous agents, such as: glutathione, Epigallacto cathecin gallate, LL37 or other cathelicidins, L-ascorbic acid, alpha-tocopherol, biotin, folic acid, methyl-salicylate, cineole, inositiol, inositol-3-phosphate, inositol-6-phosphate, L-arginine, glutamate, L-glutamine and or n-acetyl-cysteine. Anti-oxidants such as BHT, 2-hydroxybenzophenone, 2-hydroxybenzotriazole and any of the hindered amine light stabilizers may also be incorporated within the device. Natural agents for use in the bandage include, but are not limited to, A. vera WLE, 1,8 cineol, calendula officinalis extract, linalool, lavender oil derivatives, immortelle oil derivatives, camphor, white fir oil, betulin Alba terpinoids (betulinic acid), lavandula angustifolia derivatives, beta-caryophyllene, arnica, and/or non extract. Natural agents for use in the invention preferably carry key components of anti-oxidants, terpenoids, quinnones, peptitodgylcans, ethers, alcohols, furans, various amino-glycosides, amino-oligomers and/or vitamins (flavonoids).

A desired feature of the present bandage is to create a synthetic tissue growth system that can exchange wound exudates with proliferating and rehabilitating agents in a way that promotes keratin stratification and re-epithelialization. In addition, as with any open sore, especially burns, means of preventing infections is important. The present device contains an effective amount of methyl paraben, propyl paraben, sodium diacetate, phosphate buffered saline, borate and/or any other anti-microbial, macrolide, folate antagonist, antibiotics such as beta lactam, carbapene, fluoroquinone, polymixin, tetracycline, chloramphenicol, bacitracin, vancomycin, silver associated complex, metronidazole or anti-fungal agent that may be water or lipid soluble. These agents are effective against a variety of microorganisms such as staphylococcus, pseudomonas aeruginosa, Escherichia coli, Salmonella choleraesuis, Enterococcus faecalis, Staphylococcus aureus, Candida albicans, Aspergillus niger and other gram negative or positive or methacillin resistant strains.

In another aspect of the invention, a method of preparing a skin treatment bandage is provided. The formulation of the bandage is important to achieve the desired functionality. A preferred formulation with respect to the base formulation of the bandage is described below. Additional variations are also described.

In a preferred embodiment, the bandage is prepared by combining 6-10% w/v, preferably 6% PVA, 2-10% w/v, preferably 3% w/v PASP, 6-10% glycerol and/or glycol, propylene glycol, polypropylene glycol, ethylene glycol, triethylene glycol, polyethylene glycol or a derivative, preferably 10% glycerol. These components form a base gel solution. The base solution is first formulated by the dissolution of PVA in double distilled, de-ionized water or saline buffered solution, preferably ddH₂O which has been pre-treated with UV, methyl paraben and propyl paraben. The colloidal solution of PVA (which preferably comprises about 3 parts 86% hydrolyzed medium molecular weight and 1 part 99% hydrolyzed low molecular weight PVA) is maintained for 2-3 h at 90° C. with stirring, under a slight vacuum. Once complete dissolution has occurred, about 3% w/v glycerol or a derivative is added to the solution and the spectral UVN is absorbance is read at 288.5 nm and 671 nm to detect the presence of (CH₂CHOH)ₙ and (CH₂CHOCOCH₃)ₙ polymers constituents. The gel-solution is then allowed to cool to room temperature.

In a preferred embodiment, synthesis of PASP (polyaspartic acid) is carried out using the combined, modified methods described by Nita et al. (2006) and Fang Li et al. (2006). Initially 10% w/v L-Aspartic acid is boiled vigorously at 115° C. under pressure, pH=1.0 in de-ionized water and 85% o-phosphoric acid for 2 h until nearly all the liquid is removed. The colour of the sludge is a light tan. UV/Vis spectroscopic analysis should reveal a small peak at 225 nm indicating carbonyl formation and loss of the carboxylic groups. After 2 hr of boiling and stirring, a thick tan powder forms and is then washed with 70% ethanol under filter and suction before being heated at about 210° C. for 120 min. After heating the powder (polysuccinimide), a tan or beige colour should appear and as a test, should not be soluble in water (solubility: 1 g/L Bayer MSDS) (pH=3). The powder is then left to dry in a fume hood for 10 h. After drying the PSI is added to a flask with (10 mL per 2 g) of de-ionized water heated to 50° C. for 30 min (pH=3.0). The solution is then titrated with 1M NaOH (requiring approximately 0.75 molar equivalence) to a pH of about 10. During the titration, the solution, originally a cloudy beige solution, becomes a translucent red (auburn) solution. The transition typically occurs around pH 7.5. The solution is then equilibrated at 10° C. for 20 min and subsequently back titrated with 10M HCl to pH 7. Methanol (approximately 3:5 methanol:solution v/v) is then added to precipitate out the polyaspartic acid in a 2:1 v/w methanol:polysuccinimide (maximum, 2.5:1) ratio. While stirring, 10M HCl is added to the solution and polysaspartic acid is fully precipitated out at pH 2-2.5. The resultant solution is a translucent yellow colour with a white (off-white) powder (PASP sodium salt) which can be further filtered by suction.

In a modified approach, a reaction vessel can be used. While being cooled on ice or by refrigeration, 2 g PSI per 1.5 g of NaOH is stirred into approximately 10 mL of water (per 2 g PSI) for 20 min until homogenous. The solution is then back titrated to pH 7 using HCl. An off-white precipitate is formed with the addition of MeOH.

The powder is filtered and washed with methanol until a red appearance is evident. The resultant dry powder is polyaspartic acid, pH=8 and is completely soluble in water (this can be confirmed with mass spectroscopy or H NMR). The dry polyaspartic acid is then solublized in the 10% w/v PVA/3% glycerol solution at 40° C. and is mixed for about 30 min under pressure until translucent. Once the new yellow translucent gel is formed, it is cooled under a vacuum for 1 hr to remove any trapped air bubbles. This gel solution is the base component for the cryogels and produces a unique single peak at A230 nm unlike the PVA solution.

The second step of the gel synthesis can be performed in a number of ways depending upon the desired outcome of the final product. In one embodiment, the base solution is heated in water bath until it has reached 40° C., at which point the active agents may be added. An example of a preferred formulation comprises: 5% w/v L-ascorbic acid, 5% v/v alpha tocopherol, 5% v/v A. vera WLE, 5% w/v glutathione, 5% v/v cineol and 10% v/v glycerol in a base of 65% v/v PVA:PASP gel IPN. This mixture is heated until all the parts have been completely solvated by the gel. A solution of 20% borax in UV treated, de-ionized water that contains 1% sodium diacetate is prepared. This solution will complete the mixture of the gel as a 10% borax solution and 90% gel network, bonding borax and PVA with a 4:1 PVA:borax percent weight respective ratio. The finalized gel is frozen at −20° C. for 24 hr and then thawed at 25° C. The thawed solution can be poured on to casting plates and chilled (5° C.) under a slight vacuum for 1 hr or left under a vacuum at room temperature for 2-3 hr. The resulting polymer can then either be heated for 15-30 min at 200° C. to cast a translucent, thermally cross-linked polymer with a characteristic Young's modulus between 85 and 95 MPa and/or frozen and thawed up to five times at −20° C. for an additional two hours to form a cryogel matrix that will remain cold (18-22° C.) for up to 5 hr after use. Either finishing method will produce a polymer that can deliver molecular agents to the wound site, remain cooler than body surface temperature and absorb exudates up to 500% of its dry weight. It may be advantageous to incorporate certain protein complexes or cell adhesion substrates, such as biotin, into the device. These agents can be added to the cryogel surface through acid/base block-copolymerization with the reactive aspartate residues or by surface layer entrapment. Other molecules such as IL-1α, IGF and EGF may be added as a coating to the cryogel, followed by a subsequent coating of gel solution and cooled under a vacuum until solid (rubber-like). Other natural agents, essential oils, anti-oxidants or anti-microbials may be added in part to the gel solution provided that the total composition of the gel solution comprises at least 6% PVA and 2% PASP. If it is desirable to increase the amount of additives, additional agents can be layered onto the surface of the gel between freeze thaw cycles. This will ensure that the stability of the gel remains, even if it is cured by heating at 200° C. Moreover, in a similar method, the base gel may be cross linked (esterified in acid) at 200° C. for 25 min prior to incorporating the additive. The natural products can then be added through diffusion into the gel via a solvent/osmotic diffusion system.

Molecular diffusion of the active agents indicates a gel having a greater than 100 nm pore size (characterized by the radius translational motion path using the Stokes-Einstein Equation), which is appreciable for cell adhesion, proliferation and to prevent the growth of anaerobic bacteria or mold. The agents were found, using FT-PGSE RAW-BPP-LED WATERGATE H¹ NMR to diffuse through the gel at size respective rates wherein the heat-cured gel had a decreased pore size and the freeze/thaw cryogel had greatest diffusion. Nevertheless both gels were able to absorb fluid and to release the entrapped molecular agents from the gel network. Gel diffusion constants for 1,8 cineol (key ingredient) were in the order of 2.44*10⁻¹² m/s².

The gel network preferably comprises about 6-10% w/v PVA; 2-5% w/v PASP; 10-12% w/v glycerol, 4% w/v borax and approximately 3-5% w/v 1,8 cineol, 3-5% w/v A. vera WLE, 3-5% w/v calendula officinalis extract, 3-5% w/v L-ascorbic acid, 3-5% w/v alpha tocopherol, 3-5% w/v glutathione, 3-5% w/v lavender oil extract, 3-5% w/v immortelle extract, 3-5% w/v camphor, 3-5% betulin alba terpinoid extract, 3-5% w/v white fir extract, 3-5% w/v noni, 3-5% w/v inositol or inositol-3-phospate or inositol-6-phosphate, 3-5% w/v arginine, 3-5% w/v n-acetyl cysteine, <1% w/v biotin, <1% glutamate, 1-3% w/v IGF, 1-3% w/v, EGF, 1-3% w/v IL-1a and/or 3-5% w/v of an anti-microbial, anti-fungal or antibiotic as mentioned previously. The above may all be dissolved within the appropriate amount of double distilled, deionized water that has been UV treated and contains anti-fungal and anti-bacterial agents such as methyl paraben, propyl paraben and sodium diacetate.

Antioxidant therapy has shown promising results in burn victims. There are lower lipid peroxides, inflammatory responses, edema and extensive tissue loss. However, prior to the present invention the compounds used, such as vitamin C, have only been administered orally. Oral administration of agents for treatment of burns must rely on metabolic processes and a strong lymphatic and vasculature network into the burn site, both of which most burn patients do not have. The present invention provides a novel design to incorporate in its formulation, a highly effective throughput of anti-oxidants that can be delivered to the burn site. Glutathione, one of the body's best radical scavengers is readily taken up by cellular trafficking. Ascorbic acid and vitamin E, alpha tocopherol, in combination will pair to exchange radicals for export. A. vera and other natural agents provide a wide range of chelators and quinnones to effectively traffic free radical species of all types. The present device can be formulated to deliver these agents directly to the burn.

Moreover, inflammation (edema) and ischemic responses are associated with over production of wound exudates and the breakdown of supporting lymphatic and vasculature systems. The hydrogel bandage of the invention is not only able to remove exudates, but also soothes inflamed tissue by acting as a cryotherapeutic device and by compressing the tissue. In addition to being hydrophilic and cold, additives of 1,8 cineol, camphor, Lavender oil, the terpenoids and calendula officialis have been shown in-vitro to reduce inflammatory effects. Specifically 1,8 cineol has been found to reduce prostaglandin PGE2 and decrease macrophage aggregation. BRK-1 has been linked to PGE-2 and found to over be expressed in burn sites. A. vera, a natural burn agent, comprises various quinnones, polyaminoglycans and peptidylglycans that can be attributed to its burn soothing function. Calendula and any of the terpenoids are natural products that have analgesic and anti-inflammatory properties.

It is well known that despite all cellular efforts, without adhesion sites, fibroblast migration is difficult and once it is attained it can be difficult to control. Hypertrophic scarring may occur by over-stratification. The present device preferably provides a reverse basal layer system to allow inward growth of the migrating keratinocytes on the circumference of the burn.

The present invention provides therapeutic support and relief, and acts as a barrier to prevent infection. It also arrests continuous thermal insult to the tissues of burn patients immediately after the injury. These effects are achieved by the biochemical makeup of the hydrogel. These effects have been demonstrated in vitro using an innervated tissue model and also in in vivo studies. The gel is permeable for transfer of small molecules and has been proven stable and practical for the support of growing epidermal human cells in culture

The chemical synthesis of PASP produces a low molecular weight polymer using previous methods of L-aspartic acid thermal polycondensation with an acid catalyst such as O-phosphoric acid, followed by ring opening hydrolysis to produce a homopolymer of alpha and beta linkages.

While other hydrogels may deliver drugs and metabolites transdermally, the present invention has several advantages and functions by cooling surface tissue, absorbing exudates and delivering an essential variety of cell rescuing agents. The cooling effect reduces pain and inflammation. The cooling effect is driven by the evaporation of water (exudate). The device also provides a compression layer (compress dressing). The PVA:PASP polymeric IPN is esterified. The PVA:PASP polymeric IPN will form either a soft cryogel, translucent, elastomer of moderate tensile strength or a strong rubber-like semi-translucent, elastomer of high tensile strength (90 MPa). The PVA:PASP polymeric IPN of the invention has a swelling ratio (Q) greater than 500% dry weight. The PVA:PASP polymeric IPN is sufficiently porous to exchange a variety of therapeutic agents and provide breathability to the wound without possibility to draw in contaminants and infectious agents. The PVA:PASP polymeric IPN may also contain natural therapeutic agents, synthetic endogenous agents and biologics as mentioned previously that specifically enhance cell recovery in the burn site. The device, with the use of these agents will have several beneficial effects such as: (a) decrease free radical, reactive oxygen species, nitric oxide reactive species and free radicals; (b) promote cellular adhesion and differentiation; (c) supports immunological responses; (d) decreases interstitial pressure; (e) reduce ischemia and excess edema; (f) decrease necrotic and apoptotic events through reduction in TNF-alpha, FAS-L and ILK signaling, p53 induced apoptotic events, IL-8 and granule formation; (g) control Ca²⁺ influx and membrane depolarization; (h) decrease membrane permeability; (i) provide a replenishment of natural scavengers and proliferative agents; (j) provide an architecture for cellular adhesion; (k) provide a porous barrier to prevent possible infection; and (I) provide a temperature reduction which will slow peripheral vascular flow (reducing systemic release of cytokine) and reaction rates.

The device can also be designed to provide easy access for health care professionals to monitor wound re-epithelialization and minimize removal time. The device comprises two key components: (i) PVA and (ii) PASP that make it analogous to histological structures. The device is self-adhesive and is non-toxic.

While the device of the invention has been described in detail for the treatment of burns, it is clearly apparent that it can also be used for the treatment of other skin injuries.

The device may be used for burns, thermal injuries of 1^{st}, 2^{nd} and 3^{rd} degree, lacerations, abrasions, ulcers, post surgical tissues, surgically implanted devices and it may include antibiotics or other agents for delivery.

One or more currently preferred embodiments have been described by way of example. It will be apparent to persons skilled in the art that a number of variations and modifications can be made without departing from the scope of the invention as defined in the claims.

The above disclosure generally describes the present invention. It is believed that one of ordinary skill in the art can, using the preceding description, make and use the compositions and practice the methods of the present invention. A more complete understanding can be obtained by reference to the following specific examples. These examples are described solely to illustrate preferred embodiments of the present invention and are not intended to limit the scope of the invention. Changes in form and substitution of equivalents are contemplated as circumstances may suggest or render expedient. Other generic configurations will be apparent to one skilled in the art.

### EXAMPLES

Although specific terms have been used in these examples, such terms are intended in a descriptive sense and not for purposes of limitation. Methods of chemistry referred to but not explicitly described in the disclosure and these examples are reported in the scientific literature and are well known to those skilled in the art.

### Example 1

### Synthesis Method 1

A solution of 10% PVA w/v is heated to 80° C. An amount of finely ground 5% w/v of PASP is added to the PVA and mechanically stirred at 80° C. for 2 h under acidic conditions using aliquots of 10M HCl to reduce the pH to 3. After 2 h the solution pH should be approximately 6 and partial esterification of PASP with PVA will increase the viscosity and hydrophobic of the resin. The PVA:PASP is then cooled to 30° C. and the antioxidants and natural products were added in appropriate amounts totaling no more than 15% w/v of the entire gel. The resin with additives is stirred at 30° C. for 30 min. The very viscous resin is then poured into a mold (dish) and placed in a vacuum at 30° C. After 4 h the mold is removed and frozen at −80° C. and thawed for 3 cycles, after which the gel is removed from the mold and lightly sprayed with a solution of 10% w/v borax, 10% glycerol w/v and 1% cineol w/v, to cross link via H-boding the surface residual PVA hydroxyl groups. The solid gel is now dried with cool air until surface liquid is 90% removed. The resulting gel is translucent (more-so transparent) and with a cloth like texture. The gel is cold and has elasticity similar to that of rubber.

### Example 2

### Synthesis Method 2

A solution of 10% PVA w/v is heated to 80° C. An amount of finely ground 2% w/v PASP is added to the PVA and mechanically stirred at 80° C. for 2 h with pH adjusted to approximately 3 with 10M HCl. After 2 h the solution pH should be approximately 5.0 and partial esterification of the PVA with PASP should occur. The now PVA:PASP resin is cooled to 30° C. and appropriate amounts of antioxidants and natural products are added to the resin and mixed for 30 min at 30° C. The resin is poured into a mold and frozen and cooled to room temperature (21° C.), before being placed in a freezer at −80° C. The resin is then frozen and thawed 3 times, prior to being immersed in a 10% w/v borax, 10% w/v glycerol and 1% w/v cineol solution for 15 min until the gel is homogeneous and opaque white. The texture of this gel is very similar to that of natural rubber, yet is cold to the touch (18° C.).

### Example 3

### Synthesis Method 5

A solution of 10% PVA w/v is heated to 80° C. An amount of finely ground 10% w/v PASP is added to the PVA and mechanically stirred at 80° C. for 2 h with pH adjusted to approximately 2 with 10M HCl. After 2 hr the solution is poured on a glass substrate and cured at 150° C. for 30 min to produce an opaque thin film. The film is then removed and washed 3 times with solutions of 70% MeOH and EtOH. The film is then placed in saturated solution of 15 mL 5% w/v per ingredient (max of 20% w/v for total additives) per 4 cm² gel in a closed container until the liquid is removed and fully absorbed by the bandage. The bandage now partially hydrated is cool and moist, with a natural rubber texture.

### Example 4

### Synthesis Method 4

A solution of 10% PVA w/v is heated to 80° C. An amount of 10% w/v finely ground PASP is added to PVA and mechanically stirred at 80° C. for 2 hr at pH of 3. After 2 hr the PVA:PASP resin is continued to be heated at 80° C. under a vacuum for 30 min until expanded foam results. The foam is then cooled to 21° C. and poured onto a plate and allowed to dry. The resulting foam is soft and elastic like, and can be used to absorb 10% PVA resin with added natural products and subsequently cross linked with 10% borax solution for gellation.

### Example 5

### Synthesis Method 5

A solution of 10% PVA w/v is heated to 80° C. An amount of 5% w/v of finely ground PASP is added to the PVA and mechanically stirred at 80° C. for 2 hr at pH 3. After 2 hr the PVA:PASP resin should be nearest a pH of 6. It is then cooled to 30° C. and additives are mechanically stirred into solution. After 20 min of equilibrium the solution is cooled further to 21° C., poured onto a plate and via 3 freeze/thaw cycles forms a thicker resin. The resin is then placed under a vacuum for 1-2 h and is then heated for 20 min at 80° C. (This allows for curing without bubbling. If possible the resin may be heated while under a vacuum at 80° C.) The resultant gel can then be covered by a thin layer of PVA resin and subsequently frozen at −80° C. Once frozen a solution of 10% w/v borax and 10% w/v glycerol is applied to the frozen gel using a spray or mist, thereby cross linking the outer surface. This method makes for a semi-transparent gel with a hydrated temperature of 18° C. This method also reduces will create a gel with prolonged delivery time of therapeutics as they must travel through two mediums.

The chemical synthesis of PASP produces a low molecular weight polymer using previous methods of L-aspartic acid thermal polycondensation with an acid catalyst such as O-phosphoric acid, followed by ring opening hydrolysis to produce a homopolymer of alpha and beta linkages.

### Example 6

### The Use of Calcium as a Chelator of Gelation

In another preferred embodiment the PASP:PVA cross linking occurs under 60° C. heat at acidic conditions until well mixed. The formulation requires at first the solvation of (50% w/v_{PVA}) Sodium Polyaspartate of molecular weight 800-1500 Da into distilled water and acidified with 85% O-phosphoric acid or ascorbic acid to a pH of 2.5. In a separate flask, PVA at 15% w/v is dissolved into distilled watered at 60° C. until homogeneous. Calcium Chloride (0.145 g/10 mL solvent) is dissolved into the PVA solution and stirred until homogeneous. The resultant mixture is then titrated to pH 2 using 85% O-phosphoric acid. Acidic polyaspartate solution is slowly added dropwise to the PVA/CaCl₂ solution while stirring under 60° C. heat. The solution is stirred under vacuum in a rotary evaporator for 20-30 min (depending upon the volume) or until viscous). The resultant resin is then mixed with 2% v/v A. Vera whole leaf extract (gel) and mixed under vacuum at room temperature (21° C.) for 15 minutes (depending upon the volume). This resin (yellowish tint) is then incubated at 37° C. for 12 h until solid. After 8 hours the plastic-like material is washed with MeOH/EtOH/water (1:2:7) and is then placed in a buffered saline at pH 7. The now gel is swollen to 500-600% dry weight and equilibrated at pH 7. The gelled substrate can be coated with a borax crosslinked resin on the bottom (or all round) which contains natural compounds, antiseptics, antioxidants, analgesics and anti-inflammatories as described previously within this document. Furthermore, the gel may be dehydrated and rehydrated in a solution containing 5-10% w/v of these compounds described above so as to absorb them within the gel membrane.

What is claimed is:

1. A topical application device for the treatment of a skin injury, said device comprising an interpenetrating network of polyvinyl alcohol (PVA) and polyaspertate (PASP), wherein the PVA and PASP are partially cross-linked to form hydrogen bonded network.

2. The device of claim 1 comprising about 5-10% w/v PVA.

3. The device of claim 1 comprising 6-9% w/v PVA.

4. The device of claim 1 comprising 6% PVA.

5. The device of claim 1 comprising 9% PVA.

6. The device of claim 1 comprising about 2-10% w/v PASP.

7. The device of claim 1 comprising 3-5% w/v PASP.

8. The device of claim 1 further comprising 6-10% glycerol or derivatives thereof.

9. The device of claim 1 further comprising an additive selected from the group consisting of L-ascorbic acid, alpha tocopherol, A. vera WLE, glutathione, cineol, glycerol and antimicrobial agents.

10. The device of claim 9 comprising 5% w/v L-ascorbic acid.

11. The device of claim 9 comprising 5% w/v alpha tocopherol.

12. The device of claim 9 comprising 5% w/v A. vera WLE.

13. The device of claim 9 comprising 5% w/v glutathione.

14. The device of claim 9 comprising 5% w/v cineol.

15. A method of preparing a bandage for treatment of a burn, said method comprising preparing a base gel solution of PVA and PASP, and combining the base solution with an additive selected from the group consisting of L-ascorbic acid, alpha tocopherol, A. vera WLE, glutathione, cineol and glycerol.
